(19) United States
(12) Patent Application Publication
(10) Pub. No.: US 2016/0016958 A1
(43) Pub. Date: Jan. 21, 2016

(54) Method of Labeling Flumazenil with F-18 and Separating and Purifying F-18-Flumazenil

(71) Applicant: Institute of Nuclear Energy Research, Atomic Energy Council, Executive Yuan, R.O.C., Taoyuan County (TW)

(72) Inventors: Kai-Hung Cheng, Taoyuan County (TW); Yean-Hung Tu, Taoyuan County (TW); Li-Yuan Huang, Taoyuan County (TW); Yuan-Ruei Huang, Taoyuan County (TW); Kang-Wei Chang, Taoyuan County (TW)

(21) Appl. No.: 14/333,630

(22) Filed: Jul. 17, 2014

(51) Int. Cl. C07D 487/04

(52) U.S. Cl. CPC C07D 487/04

(57) ABSTRACT

Flumazenil (FMZ) is labeled with fluorine(F)-18 to obtain F-18-flumazenil. F-18-flumazenil can be strongly combined with type-A acceptor of gamma-aminobutyric acid (GABA_{A}) in brain for tracing. The time and temperature for labeling is saved and lowered. The toxic chemical, acetonitrile, used in separation and purification can be prevented. The present invention has a simplified procedure for evaluating mental disease medicines in a short time. Moreover, time for developing medicines for treating related diseases of the central nervous system can be reduced.

### FIELD OF THE INVENTION

The present invention relates to separating and purifying flumazenil (FMZ) labeled with fluorine(F)-18; more particularly, relates to fabricating a molecule probe (F-18-flumazenil) for type-A receptor of gamma-aminobutyric acid (GABA_{A}) in the central nervous system by labeling FMZ with F-18, where ethanol replaces toxic acetonitrile (ACN) for separating and purifying F-18-flumazenil and, thus, the subsequent removal process of the toxic ACN is omitted.

### BACKGROUND OF THE INVENTION

From a number of modern nerve conduction studies, it is known that a lot of modern diseases are closely related to neurotransmitters. Over-intensified nerve signaling (hyperexcitability of nervous system) is the main reason for a lot of modern physical and mental discomforts (nerve disease). These symptoms may include epilepsy, pain, bipolar disorder, stress, depression, uneasy, insomnia, schizophrenia, anger, fear and anxiety.

Gamma-aminobutyric acid (GABA) is found to be the main inhibitory neurotransmitter, whose receptors include GABA type-A receptor (GABA_{A}), GABA type-B receptor (GABA_{B}) and GABA type-C receptor (GABA_{C}). Therein, GABA_{A} has been identified as having a rapid reaction to GABA. After GABA_{A} receives GABA, its chlorine(Cl⁻) channel is opened to allow Cl⁻ enter into neurons for reducing intracellular potential. GABA is bond with the receptor to make the neurons over-polarized for relieving or suppressing excessive excitement and intense nerve signaling, which thus makes people calm down.

Now we know nerve conduction may be malfunctioned when the GABA_{A} receptors are few or have functional defects. The reasons for the few GABA_{A} receptors or the GABA_{A} receptors having functional defects include gene mutation, traumatic brain injury, and pharmacological damage. When the GABA_{A} receptors cause nerve conduction problem, some of the neurological and psychiatric disorders, including epilepsy, anxiety disorder, Parkinson's disease and chronic pain, may happen.

FMZ is a known antagonist which competes with benzodiazepine receptor to be combined with GABA. FMZ has been widely and clinically used in diagnosis and treatment of benzodiazepine poisoning. Through the binding specificity of FMZ to GABA/benzodiazepine receptor, researchers use a radioactive derivative of FMZ, which is more sensitive than [¹⁸F]-fluorodeoxyglucose ([¹⁸F]-FDG), to track and rightly point out positions causing epilepsy. Studies have also found that patients having panic disorders have far less [¹¹C]-flumazenil combined in their brain than general people. It is also found that, by using the combining ability of [¹⁸F]-flumazenil in various cortical areas of the brain, the activity of GABA/benzodiazepine receptor can be observed. With the observation, a quantified comparison can be made to brain regions causing epilepsy or being damaged by apoplexy. These show that [¹⁸F]-flumazenil is a tracer with development potential as regarding to molecular changes in the brain and central nervous system diseases and to research and development for drugs used in treatment.

As early as in 1993, some scholars used [¹¹C]-flumazenil for positioning epileptogenic zone of patients having epilepsy (Journal of Neurology, Neurosurgery, and Psychiatry 1993; 56:615-621). In 1998, some scholars used [¹¹C]-flumazenil for angiography and found that panic disorder patients have far fewer [¹¹C]-flumazenil combined in brain than normal people (Arch gen psychiatry/vol 55, August 1998). In 1999, some scholars used [³H]-flumazenil for research and found that a mouse having anxiety disorder has significantly lower binding of [³H]-flumazenil in brain (Nature neuroscience, volume 2 no 9, September 1999). In addition, some scholars found that [¹¹]-flumazenil can be used to diagnose patients having stroke in the early stage (Stroke 31; 336-369, February 2000). However, because half-life of [³H] and [¹¹C] are too short, they are not suitable to be used in other places. Later in 2005, some scholars tried using F-18 for labeling, and obtained clear images in animal bodies (Nuclear Medicine and Biology 32; 109-116 2005; Eur J Nucl Med Mol Imaging 36; 958-965 2009; Nuclear Medicine and Biology 36; 721-727 2009). However, the labeling method still has many shortcomings. The main drawback is the entire process takes too long, including reaction time, and must use high-performance liquid chromatography (High Performance Liquid Chromatography, HPLC) for processing separation and purification of the product. Moreover, the separation and purification process is coupled with toxic chemical ‘ACN’. Sequentially, a step of ACN removal is required, like concentrating under reduced pressure or using other columns.

Hence, the prior arts do not fulfill all users' requests on actual use.

### SUMMARY OF THE INVENTION

The main purpose of the present invention is to produce a molecular probe of F-18-labeled FMZ for GABA_{A} in the central nervous system.

Another purpose of the present invention is to use F-18-flumazenil as a tracer on areas of prefrontal cortex, cortex, hippocampus and amygdala owing to its binding capacity to GABA_{A} in these areas.

Another purpose of the present invention to reduce the time and the temperature for labeling FMZ to 15 minutes (min) and 150 celsius degrees (° C.) without decreasing the yield; and to replace toxic ACN with ethanol on separating and purifying F-18-flumazenil while the subsequent removal process of the toxic ACN is omitted for easy operation with time saved.

Another purpose of the present invention is to use F-18-flumazenil to break through blood-brain barrier and obtain a high affinity to GABA_{A} in animal brains for evaluating efficacy of drugs for related psychiatric disorders, like anxiety disorder, schizophrenia, and epilepsy and for effectively shortening the schedule on developing treatment drugs for related diseases of the central nervous system.

To achieve the above purposes, the present invention is a method of labeling FMZ with F-18 and separating and purifying F-18-flumazenil, comprising steps of: (a) adding a potassium carbonate (K₂CO₃) solution, a cryptand solution, a precursor (Nitromazenil) solution, an ACN solution and an injection water into a first to a fifth tubes, respectively; (b) preparing a plurality of sixth tubes by washing with methanol once and being dried; (c) sucking 0.2 milli-liters (mL) of a F-18 (18F/H₂¹⁸O) solution with activity and dose calculated; (d) processing a fluorine-oxide (F—O) separation to the F-18 solution through an ion exchange resin to adhere F-18 on the ion exchange resin and collecting residual solution in one of the sixth tubes; (e) directing the K₂CO₃ solution to wash down F-18 adhered on the ion exchange resin into another one of the sixth tubes to obtain a labeling tube; (f) directing the cryptand solution into the labeling tube and heating the labeling tube; (g) directing the ACN solution into the labeling tube and heating the labeling tube; (h) after cooling down the labeling tube, processing blowing and sucking with nitrogen; (i) directing the precursor solution into the labeling tube and heating the labeling tube for processing reaction at a temperature between 120˜180° C. for 12˜18 min; (j) after cooling down the labeling tube, directing the injection water to dilute a product obtained from the reaction processed in step (i) and directly collecting the diluted product into another one of the sixth tubes to obtain a collecting tube; (k) separating and purifying the product in the collecting tube by using semi-preparative high performance liquid chromatography (HPLC); and (l) filtering the product, F-18-flumazenil, with a syringe filter to remove impurities and strains and storing the filtered product in a sterile tube. Accordingly, a novel method of labeling FMZ with F-18 and separating and purifying F-18-flumazenil is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the flow view showing the synthesis reactions for fabricating F-18-flumazenil;

FIG. 3 is the view showing the chemical equation of F-18-flumazenil;

FIG. 4 is the view showing the radiochemical purity of F-18-flumazenil by using Radio-TLC;

FIG. 5 is the view showing the radiochemical purity of F-18-flumazenil by using HPLC;

FIG. 6 is the view showing the processes for testing lipophilicity of F-18-flumazenil;

FIG. 7 is the view showing the stability of F-18-flumazenil; and

FIG. 8 is the view showing the nanoPET/CT images of brain of normal rat injected with F-18-flumazenil.

### DESCRIPTION OF THE PREFERRED EMBODIMENT

The following description of the preferred embodiment is provided to understand the features and the structures of the present invention.

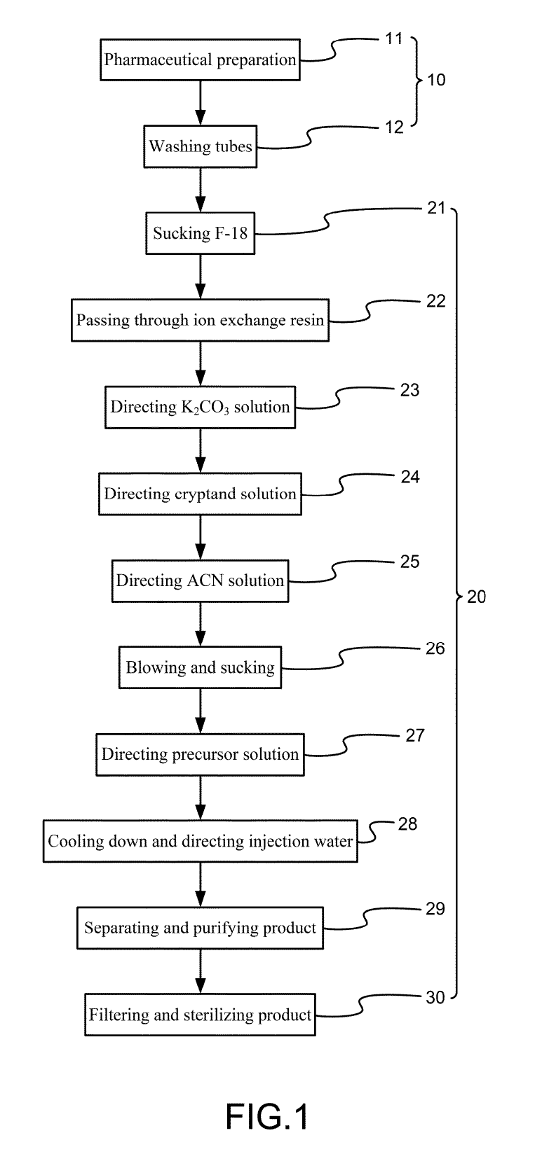

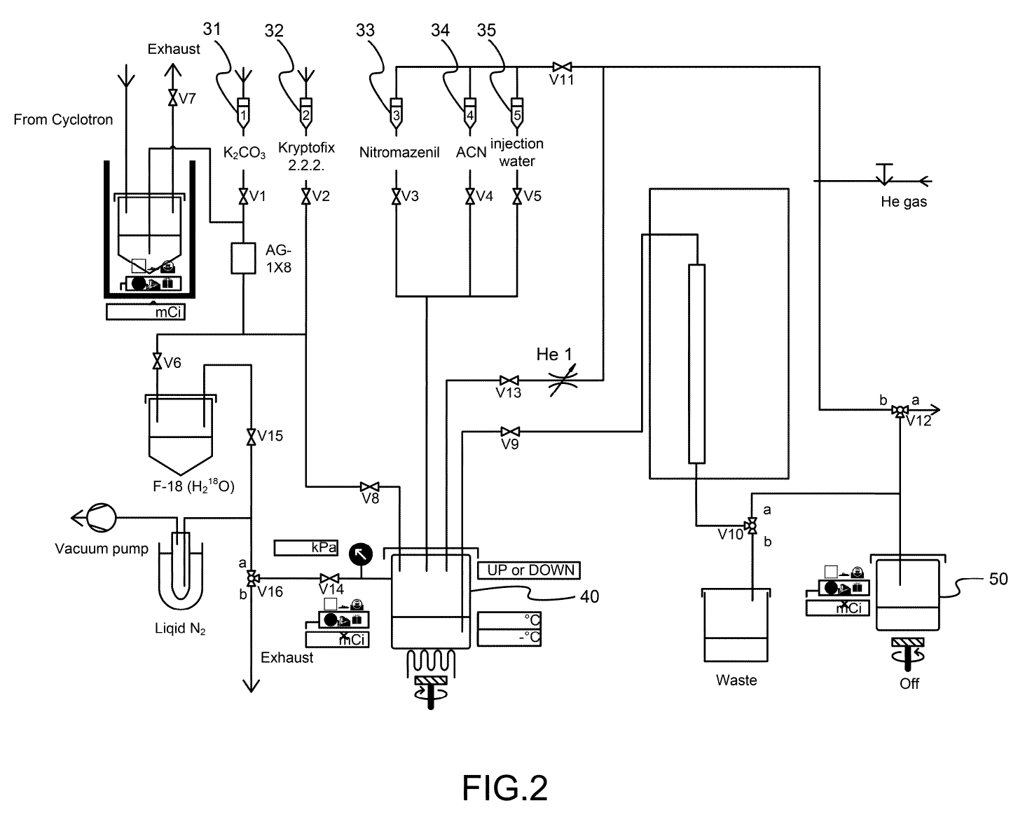

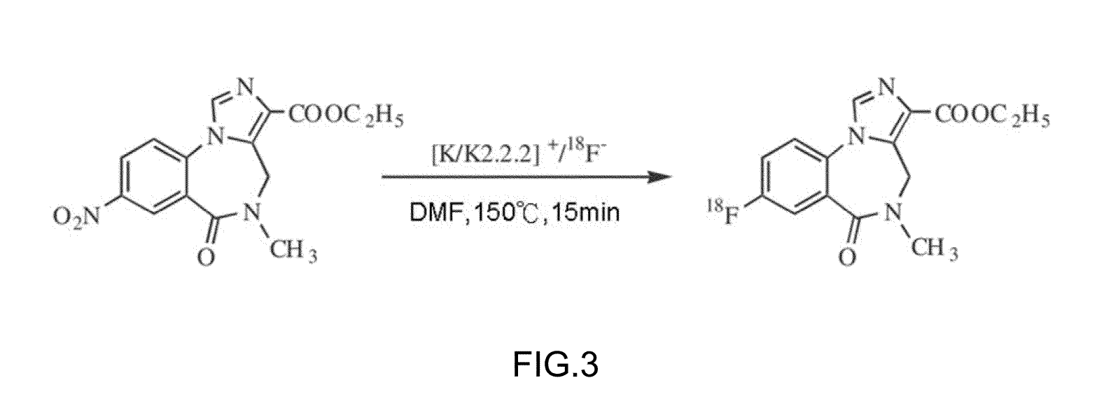

Please refer to FIG. 1 to FIG. 3, which are a flow view showing a preferred embodiment according to the present invention; a flow view showing synthesis reactions for fabricating F-18-flumazenil; and a view showing a chemical equation of F-18-flumazenil. As shown in the figures, the present invention is a method of labeling flumazenil (FMZ) with fluorine(F)-18 and separating and purifying F-18-flumazenil, comprising the following steps:

### [Preparation 10]

(a) Pharmaceutical preparation 11: The present invention uses an automatic synthesis box of GE TRACERlab FX-FN module for reactions. Drugs are added into tubes: A first tube 31 is added with 3.5 milligrams per milliliter (mg/mL) of a potassium carbonate (K₂CO₃) solution; a second tube 32 is added with 15 mg/mL of a cryptand (Kryptofix 2.2.2.) solution; a third tube 33 is added with 10 mg/mL of a precursor (Nitromazenil) solution; a fourth tube 34 is added with 1 mg/mL of an acetonitrile (ACN) solution; and, a fifth tube 35 is added with 2 mL of an injection water.

(b) Washing tubes 12: A plurality of sixth tubes are prepared by washing with methanol once and, then, being dried by air-blowing.

### [Operation 20]

(c) Sucking F-18 21: 0.2 mL of a F-18 (18F/H₂¹⁸O) solution is sucked with its activity and dose calculated.

(d) Passing through ion exchange resin 22: The F-18 solution is passed through an AG-1-X8 ion exchange resin to process a fluorine-oxide (F—O) separation for adhering F-18 on the ion exchange resin. Remaining part of the F-18 solution is collected into one of the sixth tubes for recycling;

(e) Directing K₂CO₃ solution 23: The K₂CO₃ solution in the first tube 31 is directed inward to wash down F-18 adhered on the ion exchange resin into another one of the sixth tubes to obtain a labeling tube 40.

(f) Directing cryptand solution 24: The cryptand solution in the second tube 32 is directed to the labeling tube 40 and, then, is heated to 95 celsius degrees (° C.) for about 3 minutes (min).

(g) Directing ACN solution 25: The ACN solution in the fourth tube 34 is directed to the labeling tube 40 and, then, is heated to 95° C. for about 2 min.

(h) Blowing and sucking 26: The labeling tube 40 is cooled down to 50° C. and, then, blowing and sucking are processed with nitrogen for 3 min.

(i) Directing precursor solution 27: The precursor (Nitromazenil) solution in the third tube 33 is directed to the labeling tube 40 and, then, is heated to 150° C. for reaction for about 15 min.

(j) Cooling down and directing injection water 28: The labeling tube 40 is cooled down to 50° C. and, then, the injection water in the fifth tube 35 is directed inward to dilute a product obtained after the reaction processed in step (i). The diluted product is directly collected into another one of the sixth tubes to obtain a collecting tube 50.

(k) Separating and purifying product 29: The product in the collecting tube 50 is separated and purified by using semi-preparative high performance liquid chromatography (HPLC).

(l) Filtering and sterilizing product 30: The product (F-18-flumazenil) thus obtained is filtered with a 0.22 micrometers (μm) syringe filter to remove impurities and bacteria. The filtered product of F-18-flumazenil is stored in a sterile tube. Therein, on using, a sterile normal saline is used to dilute the product for making the alcohol content of a final solution become less than 20%.

In step (k), columns used for separation and purification are 7.8×300 mm semi-preparative C18 column, Waters, with a flowing buffer of ethanol and water at a ratio of 20:80 during the previous 0˜20 min. During the later 20˜40 min, with a solution of ethanol and water at a ratio of 30:70 under a flow speed of 3 mL/min, a radiation detector of flow count is used for analysis.

In steps (l), Radio-TLC or HPLC is used for analyzing the radiochemical purity of the product of F-18-flumazenil, where the radiochemical purity of F-18-flumazenil should not be not less than 90%.

Thus, a novel method of labeling FMZ with F-18 and separating and purifying F-18-flumazenil is obtained.

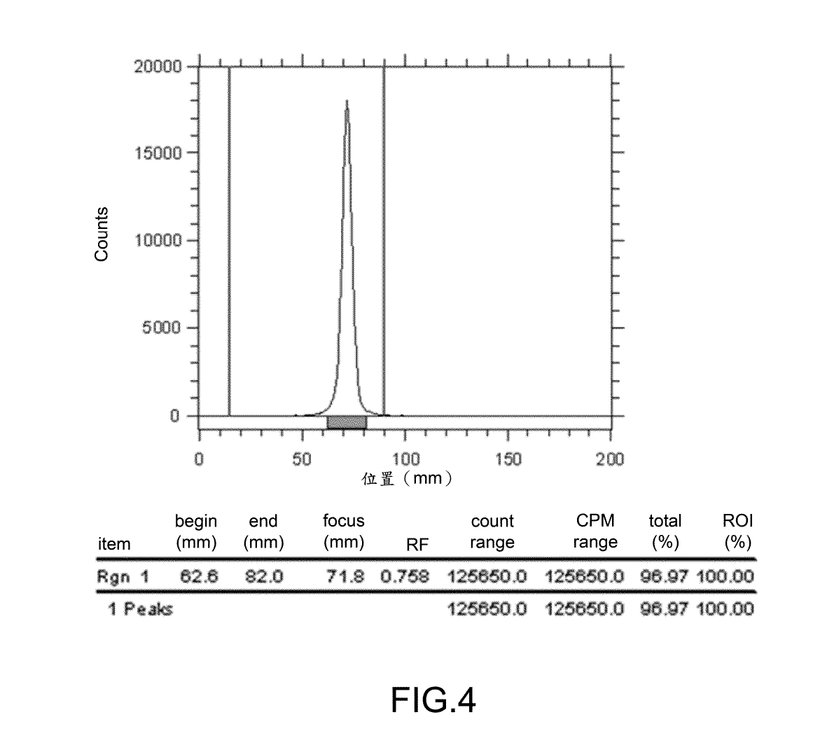

Please refer to FIG. 4, which is a view showing a radiochemical purity of F-18-flumazenil by using Radio-TLC. As shown in the figure, a product of F-18-flumazenil fabricated according to the present invention is analyzed by using Radio-TLC for obtaining its radiochemical purity. The condition for analysis comprises a 1.5×10 cm paper of Silica gel 60 F₂₅₄ for a stationary phase and a developing solution of ethyl acetate (EA) and ethanol at a ratio of 8:2 for a mobile phase. Through detection and analysis, an RF value (retention factor) as 0.7 and a radiochemical purity greater than 90% are obtained for the product of F-18-flumazenil.

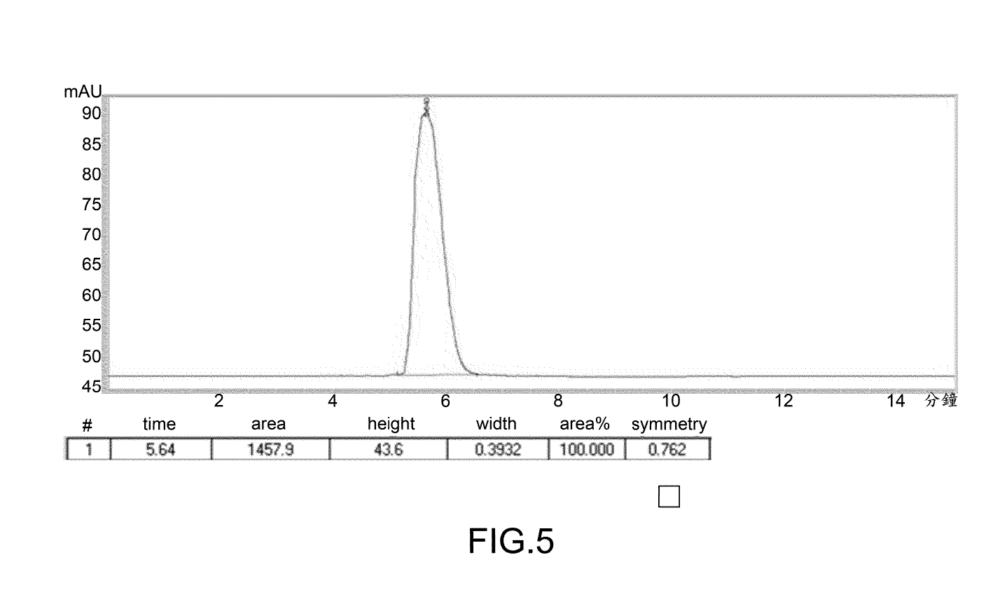

Please refer to FIG. 5, which is a view showing a radiochemical purity of F-18-flumazenil by using HPLC. As shown in the figure, a product of F-18-flumazenil fabricated according to the present invention is analyzed by using HPLC, which is equipped with a radioactivity detector, for obtaining its radiochemical purity. Chromatographic columns used are 3.9×150 mm C18 columns. The condition for chromatographic analysis comprises an eluent of ACN and 0.01 M phosphate at a mixing ratio of 30:70 under a flow speed of 1 mL/min to be compared with an FMZ authentic product. Through detection and analysis, a residence time about 5 min and a radiochemical purity greater than 90% are obtained for the product of F-18-flumazenil.

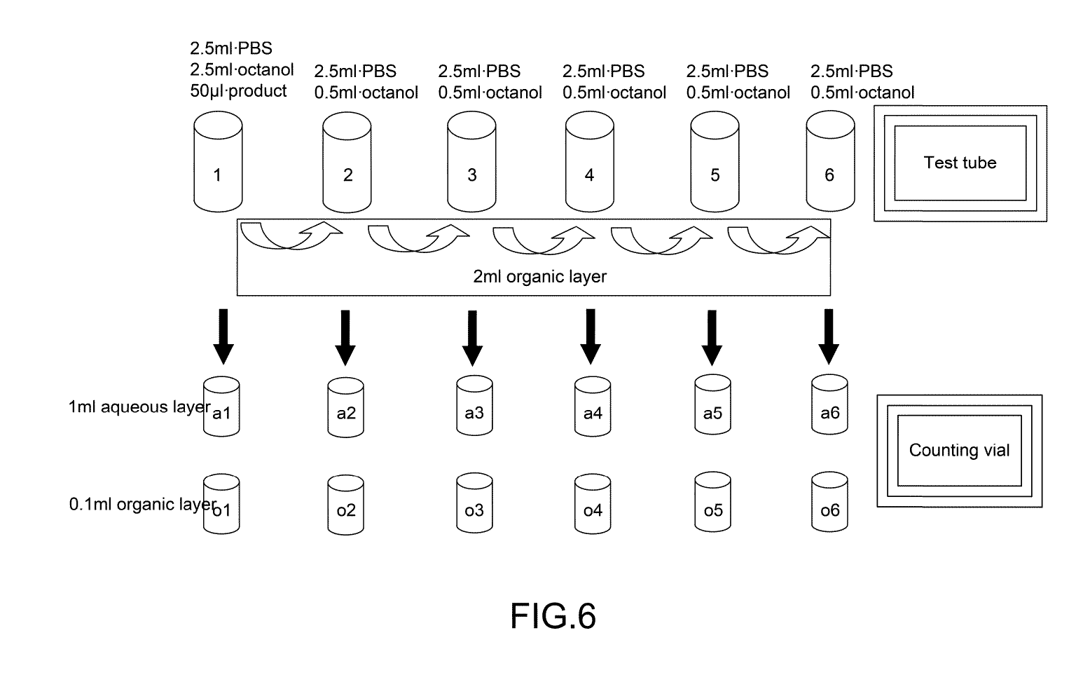

Please refer to FIG. 6, which is a view showing processes for testing lipophilicity of F-18-flumazenil. As shown in the figure, for testing lipophilicity of F-18-flumazenil, an analysis to proportions of hydrophilic phosphate buffer saline (PBS) and lipophilic octanol is done. 50 micro-liters (μl) of F-18-flumazenil is mixed with 0.5 ml of PBS and 0.5 ml of octanol. Then, an octanol phase is diluted to obtain an octanol-phase solution together with an equivalent amount of a water-phase solution. A gamma counter is used for calculating a log P value through the following formula: Log P=Log {(Decay corrected activity)organic layer×10/(Decay corrected activity)aqueous layer}. Therein, the log P value represents the lipophilicity of F-18-flumazenil.

Through the calculation, the log P value of F-18-flumazenil is 1.49±0.12, which clearly shows high lipophilicity and, therefore, can easily pass through the blood-brain barrier (BBB).

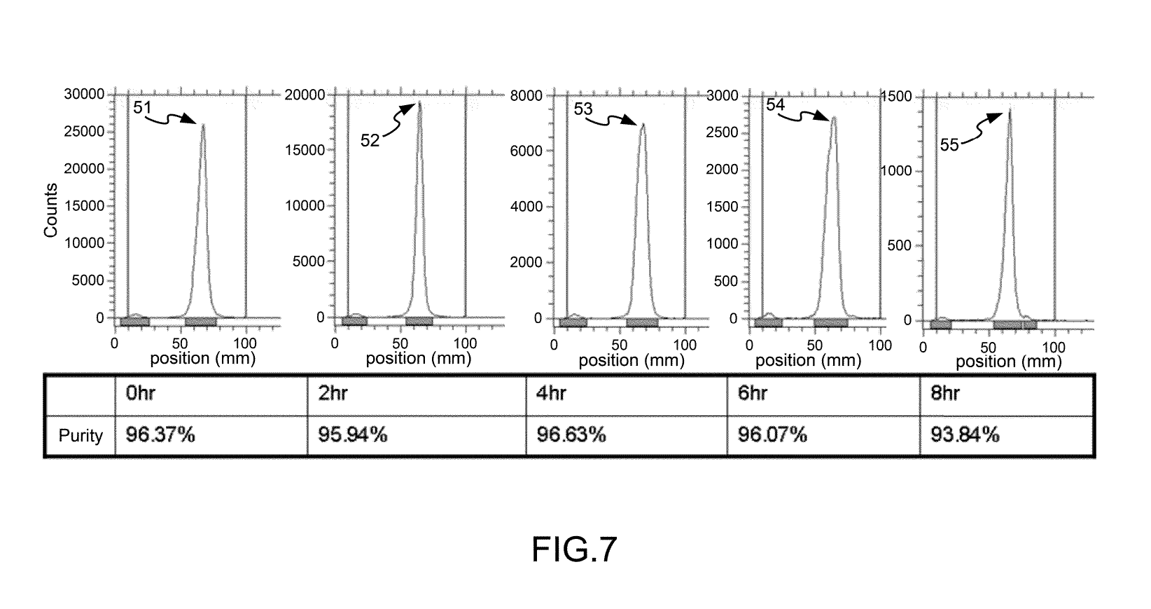

Please refer to FIG. 7, which is a view showing stability of F-18-flumazenil. As shown in the figure, after a product of F-18-flumazenil is stayed still at a room temperature for 0, 2, 4, 6 and 8 hours, radiochemical purity is measured. As a result shown with liquid peaks 51,52,53,54,55, F-18-flumazenil remains its stability greater than 90%.

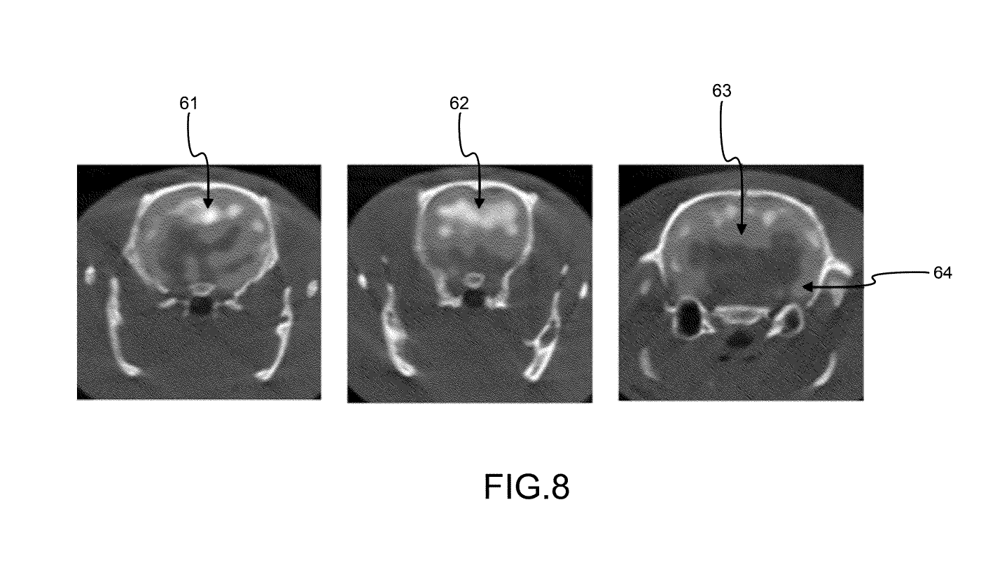

Please refer to FIG. 8, which is a view showing nanoPET/CT images of brain of normal rat injected with F-18-flumazenil. As shown in the figure, 1 mCi of F-18-flumazenil is injected for imaging brains of normal rats through nanoPET/CT. After comparing with each other, obvious intake doses are found in coronal areas of cortex region 61, prefrontal cortex region 62, hippocampus region 63 and amygdala region 64. Thus, it is confirmed that F-18-flumazenil can enter animal brain to be used as an imaging agent for type-A receptor of gamma-aminobutyric acid (GABA_{A}) in the central nervous system (CNS). Hence, F-18-flumazenil can be applied for evaluating effectiveness of CNS-related-disease drugs.

Conclusively, F-18-flumazenil fabricated according to the present invention has the following characteristics and effectiveness:

1. It is found that images taken at 15, 30, 45 and 60 min have no big difference in between. Hence, the response time is shortened to 15 min.

2. Although HPLC is still used in the subsequent separation and purification process, ACN is replaced with ethanol and, hence, there is no need for water dilution with SPE column. The product fabricated according to the present invention simply needs to dilute ethanol to an acceptable concentration range. The final product yield with the use of ethanol for separation and purification is approximately 18.14±2.98%. Moreover, ACN is a toxic chemical substance which would results in environmental pollution and harming human health. The use of low toxic ethanol avoids residual harmful substance produced in subsequent cleanup procedure.

3. As comparing with the prior arts, although an automated synthesis cartridge is also used, the present invention reduces the reaction time to 15 min, which is shortened for about 70 min. Not to mention that the subsequent process for removing ACN is omitted with the whole operation made easier.

4. In the images of brain of normal rats, it is confirmed that F-18-flumazenil fabricated according to the present invention can pass through BBB and has high binding capacity to GABA_{A} in CNS. Hence, F-18-flumazenil fabricated according to the present invention can be applied for evaluating effectiveness of drugs for CNS-related diseases, like anxiety disorder, schizophrenia and epilepsy, with drug-developing time effectively shortened.

To sum up, the present invention is a method of labeling FMZ with F-18 and separating and purifying F-18-flumazenil, where F-18-flumazenil fabricated according to the present invention achieves a labeling yield of 18.14±2.98% with a radiochemical purity more than 90%; the present invention not only has a short reaction time, a high yield, but also provides a simple process for medical brain tomography with F-18-flumazenil; and, the present invention effectively reduces harm to pharmaceutical operation personnel by reducing time for being exposed under radiation.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of labeling flu mazenil (FMZ) with fluorine(F)-18 and separating and purifying F-18-flumazenil, comprising steps of:
(a) adding a potassium carbonate (K₂CO₃) solution, a cryptand (Kryptofix 2.2.2.) solution, a precursor (Nitromazenil) solution, an acetonitrile (ACN) solution and an injection water into a first to a fifth tubes, respectively;
(b) preparing a plurality of sixth tubes by washing with methanol once and being dried;
(c) obtaining 0.2 milli-liters (mL) of a F-18 (18F/H₂¹⁸O) solution with activity and dose calculated;
(d) processing a fluorine-oxide (F—O) separation to said F-18 solution through an ion exchange resin to adhere F-18 on said ion exchange resin and collecting residual solution in one of said sixth tubes;
(e) directing said K₂CO₃ solution in said first tube to wash down F-18 adhered on said ion exchange resin into another one of said sixth tubes to obtain a labeling tube;
(f) directing said cryptand solution in said second tube into said labeling tube and heating said labeling tube;
(g) directing said ACN solution in said fourth tube into said labeling tube and heating said labeling tube;
(h) after cooling down said labeling tube, processing blowing and sucking with nitrogen;
(i) directing said precursor solution in said third tube into said labeling tube and heating said labeling tube to process reaction at a temperature between 120˜180 celsius degrees (° C.) for 12˜18 minutes (min);
(j) after cooling down said labeling tube, directing said injection water in said fifth tube to dilute a product obtained after said reaction processed in step (i) and directly collecting said diluted product into another one of said sixth tubes to obtain a collecting tube;
(k) separating and purifying said product in said collecting tube by using semi-preparative high performance liquid chromatography (HPLC); and
(l) filtering said product, F-18-flumazenil, with a syringe filter to remove impurities and bacteria and storing said filtered product, F-18-flumazenil, in a sterile tube.

2. The method according to claim 1,
wherein, in step (d), said ion exchange resin is an AG-1-X8 ion exchange resin.

3. The method according to claim 1,
wherein, in step (f), a reaction is processed at a temperature between 76˜114° C. for 2˜4 min.

4. The method according to claim 1,
wherein, in step (g), a reaction is processed at a temperature between 76˜114° C. for 1˜3 min.

5. The method according to claim 1,
wherein, in step (h), after cooling down said labeling tube to 40˜60° C., blowing and sucking are processed with nitrogen for 2˜4 min.

6. The method according to claim 1,
wherein, in step (j), said labeling tube is cooled down to 40˜60° C.

7. The method according to claim 1,
wherein, in step (k), a semi-preparative C18 column is used; at first, ethanol and water at a ratio of 20:80 is used as a flowing buffer for 0-20 min; then, ethanol and water at a ratio of 30:70 is used as said flowing buffer for next 20 min; and, under a flow speed of 2˜4 milliliters per minutes (mL/min), a radiation detector of flow count is used to process analysis.

8. The method according to claim 1,
wherein, in step (l), said syringe filter has a filtering size of 0.15˜0.25 micrometers (μm).

9. The method according to claim 1,
wherein said product, F-18-flumazenil, has a labeling yield of 18.14±2.98%, and has a radiochemical purity greater than 90%.
